(19) United States
(12) Patent Application Publication
(10) Pub. No.: US 2011/0251481 A1
(43) Pub. Date: Oct. 13, 2011

(54) Piezoelectrically-Driven Power Injector

(76) Inventor: Geoffrey S. Strobl, Williamsburg, OH (US)

(21) Appl. No.: 13/139,544

(22) PCT Filed: Oct. 11, 2010
(86) PCT No.: PCT/US10/52142

§ 371 Date: Jun. 14, 2011

Related U.S. Application Data
(60) Provisional application No. 61/251,773, filed on Oct. 15, 2009.

(51) Int. Cl. A61B 5/055; A61M 5/31; A61B 6/00

(52) U.S. Cl. 600/420; 600/432

(57) ABSTRACT

A syringe plunger drive assembly (132) for advancing a syringe plunger (90a/b) in at least one direction (e.g., to deliver fluid). In one embodiment, this syringe plunger drive assembly (132) may be constructed of all non-ferrous components and includes a piezoelectric pump (110) that supplies pressurized hydraulic fluid to a syringe plunger driver (134). The syringe plunger driver (134) may be in the form of a single acting hydraulic cylinder including a piston rod (140) movable in first and second generally opposing directions (e.g. forward and rearward). A valve (152) and a reservoir (154) may be fluidly interconnected between the piezoelectric pump (110) and the syringe plunger driver (134) to facilitate the pumping of hydraulic fluid to the syringe plunger driver (134).

### CROSS-REFERENCES TO RELATED APPLICATIONS

This application claims priority to and the benefit of U.S. Provisional Application Ser. No. 61/251,773 entitled “PIEZOELECTRICALLY-DRIVEN POWER INJECTOR” filed on 15 Oct. 2009.

### FIELD OF THE INVENTION

The present invention generally relates to the field of medical fluid delivery systems and, more particularly, to medical fluid delivery systems with drive sources suitable to operate within a magnetic resonance imaging (MRI) environment.

### BACKGROUND

Various medical procedures require that one or more medical fluids be injected into a patient. For example, medical imaging procedures oftentimes involve the injection of contrast media into a patient, possibly along with saline and/or other fluids. Other medical procedures involve injecting one or more fluids into a patient for therapeutic purposes. Power injectors may be used for these types of applications.

A power injector generally includes what is commonly referred to as a powerhead. One or more syringes may be mounted to the powerhead in various manners (e.g., detachably; rear-loading; front-loading; side-loading). Each syringe typically includes what may be characterized as a syringe plunger, piston, or the like. Each such syringe plunger is designed to interface with (e.g., contact and/or temporarily interconnect with) an appropriate syringe plunger driver that is incorporated into the powerhead, such that operation of the syringe plunger driver axially advances the associated syringe plunger inside and relative to a barrel of the syringe. One typical syringe plunger driver is in the form of a ram that is mounted on a threaded lead or drive screw. Rotation of the drive screw in one rotational direction advances the associated ram in one axial direction, while rotation of the drive screw in the opposite rotational direction advances the associated ram in the opposite axial direction.

### SUMMARY

A first aspect of the present invention is embodied by a medical fluid delivery system that includes an injection device and at least one syringe, where each such syringe includes a syringe barrel and a syringe plunger. The injection device utilizes a syringe plunger driver (e.g., a first syringe plunger driver) and a piezoelectric pump that is operatively interconnected to the syringe plunger driver for driving the syringe plunger driver in at least a first direction. The syringe plunger is moved in at least the first direction, within and relative to its corresponding syringe barrel, by way of interaction with the corresponding syringe plunger driver.

A number of feature refinements and additional features are applicable to the first aspect of the present invention. These feature refinements and additional features may be used individually or in any combination. As such, each of the following features that will be discussed may be, but are not required to be, used with any other feature or combination of features of the first aspect. The following discussion is applicable to the first aspect, up to the start of the discussion of the term “fluidly interconnected”. The “first direction” in which a syringe plunger driver may be moved may be that which is associated with a discharge stroke of its corresponding syringe.

In an embodiment, the syringe plunger driver may include a hydraulic cylinder and in a first variation, the hydraulic cylinder may be single acting. The single acting hydraulic cylinder may include a piston, a piston rod and at least one biasing member. Any appropriate type of biasing member may be used, any appropriate number of biasing members may be used, and each such biasing member may be integrated in any appropriate manner. The at least one biasing member may be disposed to urge the piston and the piston rod in a second direction opposite the first direction. Further, the piezoelectric pump may be appropriately interconnected to the hydraulic cylinder. For instance, the piezoelectric pump may include an inlet port and an outlet port and the hydraulic cylinder may include a first port, and hydraulic fluid from the outlet port of the piezoelectric pump may enter the first port of the hydraulic cylinder to move the piston and piston rod in the first direction. The system may also include a valve movable between an at least partially open position and a closed position. Fluid may be operable to flow out of the first port of the piezoelectric pump when the valve is in the at least partially open position, and fluid may be substantially not operable to flow out of the first port when the valve is in the closed position.

In a second variation, the hydraulic cylinder may be double acting. In this variation, the piezoelectric pump may have an inlet port and an outlet port, and the hydraulic cylinder may have a first port and a second port. The system may have valving operable to selectively interconnect the inlet port of the piezoelectric pump to the first port of the hydraulic cylinder and the outlet port of the piezoelectric pump to the second port of the hydraulic cylinder in a first configuration. In a second configuration, the valving may interconnect the inlet port of the piezoelectric pump to the second port of the hydraulic cylinder and the outlet port of the piezoelectric pump to the first port of the hydraulic cylinder.

In an embodiment, the syringe plunger driver may include a hydraulic motor. In this embodiment, the syringe plunger driver may have a ram movable on a drive screw. As such, the hydraulic motor may assist in rotating the drive screw to move the ram along the drive screw to move the syringe plunger. A driving gear may be mounted to the hydraulic motor and a driven gear mounted on the drive screw such that rotation of the driving gear rotates the drive screw.

The injection device may include a housing, and the piezoelectric pump and the syringe plunger driver may be located at least partially within the housing. Additionally or alternatively, the injection device may be attached to one of a pedestal and a suspension arm. The system may include a reservoir whereby the syringe plunger driver is selectively fluidly interconnectable to the reservoir and the piezoelectric pump is fluidly interconnected to the reservoir. The system may also include at least one piezoelectric valve, although other appropriate types of valves are contemplated.

The piezoelectric pump may include a pumping chamber and a piezoelectric stack. The piezoelectric stack may be operable to expand to perform one of expelling hydraulic fluid from the pumping chamber and drawing hydraulic fluid into the pumping chamber, and to contract to perform the other of expelling hydraulic fluid from the pumping chamber and drawing hydraulic fluid into the pumping chamber. An expansion of the piezoelectric stack may serve to drive the syringe plunger driver in the above-noted first direction.

In an embodiment, the injection device may include a second syringe plunger driver, along with a corresponding second syringe having a second syringe barrel and a second syringe plunger (e.g., where the second syringe plunger is disposed within and movable relative to the second syringe barrel). The piezoelectric pump may be operatively interconnected to the second syringe plunger driver for driving the second syringe plunger driver in at least a corresponding first direction. The second syringe plunger driver may interact with the second syringe plunger of the second syringe to move the second syringe plunger in at least its corresponding first direction. In one variation, a selector valve may be operable to fluidly interconnect the first syringe plunger driver to the piezoelectric pump in a first position of the selector valve and the second syringe plunger driver to the piezoelectric pump in a second position of the selector valve. In another variation, the injection device may include a directional valve movable between first and second positions. The first position of the directional valve may be operable to fluidly interconnect an inlet port of the piezoelectric pump to a first port of one of the first or second syringe plunger drivers (e.g., as determined by the position of a selector valve) and an outlet port of the piezoelectric pump to a second port of the one of the first or second syringe plunger drivers. The second position of the directional valve may be operable to fluidly interconnect the inlet port to the second port of the one of the first or second syringe plunger drivers and the outlet port to the first port of the one of the first or second syringe plunger drivers.

In an embodiment, a magnetic resonance imaging suite may include an entirety of at least one of the systems as described above. The suite may include a room shielded from electromagnetic interference. The suite may further include at least one magnetic resonance imaging device.

As used herein, the term “fluidly interconnected” refers to two or more components or entities being connected (directly or indirectly) in a manner such that fluid can flow (e.g., unidirectionally or bidirectionally) in a predetermined flow path therebetween. For example, “an injection device fluidly interconnected to a patient” describes a configuration where fluid can flow from the injection device through any interconnecting devices (e.g., tubing, connectors) and into the patient (e.g., into the vasculature of the patient).

A number of feature refinements and additional features are separately applicable to the above-noted first aspect of the present invention. These feature refinements and additional features may be used individually or in any combination in relation to the above-noted first aspect. Any feature of any other various aspects of the present invention that is intended to be limited to a “singular” context or the like will be clearly set forth herein by terms such as “only,” “single,” “limited to,” or the like. Merely introducing a feature in accordance with commonly accepted antecedent basis practice does not limit the corresponding feature to the singular (e.g., indicating that a power injector includes “a syringe” alone does not mean that the power injector includes only a single syringe). Moreover, any failure to use phrases such as “at least one” also does not limit the corresponding feature to the singular (e.g., indicating that a power injector includes “a syringe” alone does not mean that the power injector includes only a single syringe). Finally, use of the phrase “at least generally” or the like in relation to a particular feature encompasses the corresponding characteristic and insubstantial variations thereof (e.g., indicating that a syringe barrel is at least generally cylindrical encompasses the syringe barrel being cylindrical).

Any power injector that may be utilized to provide a fluid discharge may be of any appropriate size, shape, configuration, and/or type. Any such power injector may utilize one or more syringe plunger drivers of any appropriate size, shape, configuration, and/or type, where each such syringe plunger driver is capable of at least bi-directional movement (e.g., a movement in a first direction for discharging fluid; a movement in a second direction for accommodating a loading and/or drawing of fluid and/or so as to return to a position for a subsequent fluid discharge operation), and where each such syringe plunger driver may interact with its corresponding syringe plunger in any appropriate manner (e.g., by mechanical contact; by an appropriate coupling (mechanical or otherwise)) so as to be able to advance the syringe plunger in at least one direction (e.g., to discharge fluid). Each syringe plunger driver may utilize one or more drive sources of any appropriate size, shape, configuration, and/or type. Multiple drive source outputs may be combined in any appropriate manner to advance a single syringe plunger at a given time. One or more drive sources may be dedicated to a single syringe plunger driver, one or more drive sources may be associated with multiple syringe plunger drivers (e.g., incorporating a transmission of sorts to change the output from one syringe plunger to another syringe plunger), or a combination thereof. Representative drive source forms include a brushed or brushless electric motor, a hydraulic motor, a pneumatic motor, a piezoelectric motor, or a stepper motor.

Any such power injector may be used for any appropriate application where the delivery of one or more medical fluids is desired, including without limitation any appropriate medical application (e.g., computed tomography or CT imaging; magnetic resonance imaging or MRI; single photon emission computed tomography or SPECT imaging; positron emission tomography or PET imaging; X-ray imaging; angiographic imaging; optical imaging; ultrasound imaging). Any such power injector may be used in conjunction with any component or combination of components, such as an appropriate imaging system (e.g., a CT scanner). For instance, information could be conveyed between any such power injector and one or more other components (e.g., scan delay information, injection start signal, injection rate).

Any appropriate number of syringes may be utilized with any such power injector in any appropriate manner (e.g., detachably; front-loaded; rear-loaded; side-loaded), any appropriate medical fluid may be discharged from a given syringe of any such power injector (e.g., contrast media, a radiopharmaceutical, saline, and any combination thereof), and any appropriate fluid may be discharged from a multiple syringe power injector configuration in any appropriate manner (e.g., sequentially, simultaneously), or any combination thereof. In one embodiment, fluid discharged from a syringe by operation of the power injector is directed into a conduit (e.g., medical tubing set), where this conduit is fluidly interconnected with the syringe in any appropriate manner and directs fluid to a desired location (e.g., to a catheter that is inserted into a patient for injection). Multiple syringes may discharge into a common conduit (e.g., for provision to a single injection site), or one syringe may discharge into one conduit (e.g., for provision to one injection site), while another syringe may discharge into a different conduit (e.g., for provision to a different injection site). In one embodiment, each syringe includes a syringe barrel and a plunger that is disposed within and movable relative to the syringe barrel. This plunger may interface with the power injector's syringe plunger drive assembly such that the syringe plunger drive assembly is able to advance the plunger in at least one direction, and possibly in two different, opposite directions.

### BRIEF DESCRIPTION OF THE FIGURES

FIG. 1 is a schematic of one embodiment of a power injector.

FIG. 2A is a perspective view of one embodiment of a portable stand-mounted, dual-head power injector.

FIG. 2B is an enlarged, partially exploded, perspective view of a powerhead used by the power injector of FIG. 2A.

FIG. 2C is a schematic of one embodiment of a syringe plunger drive assembly that may be used by the power injector of FIG. 2A.

FIG. 2D is a schematic of one embodiment of a syringe plunger drive assembly that may be used by the power injector of FIG. 2A and that includes a single acting hydraulic cylinder and a piezoelectric drive source.

FIG. 2E is a schematic of one embodiment of a syringe plunger drive assembly that may be used by the power injector of FIG. 2A and that includes a double acting hydraulic cylinder and a piezoelectric drive source.

FIG. 2F is a schematic of one embodiment of a syringe plunger drive assembly that may be used by the power injector of FIG. 2A and that includes two double acting hydraulic cylinders and a piezoelectric drive source.

FIG. 2G is a schematic of one embodiment of a syringe plunger drive assembly that may be used by the power injector of FIG. 2A and that includes a hydraulic motor and a piezoelectric drive source.

FIG. 3 is a schematic of one embodiment of an MRI suite having a power injector that may use any of the piezoelectrically-driven hydraulic syringe plunger drive assemblies of FIGS. 2D-2F.

### DETAILED DESCRIPTION

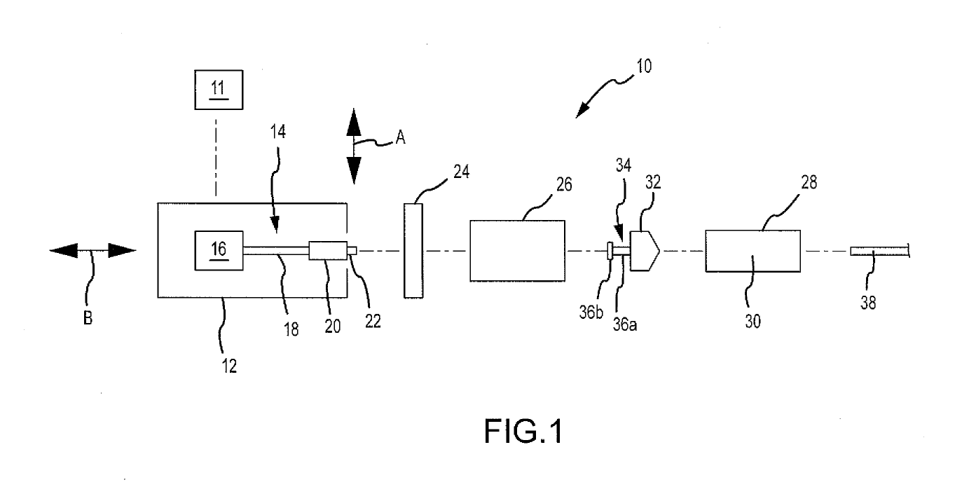

FIG. 1 presents a schematic of one embodiment of a power injector 10 having a powerhead 12. One or more graphical user interfaces or GUIs 11 may be associated with the powerhead 12. Each GUI 11: 1) may be of any appropriate size, shape, configuration, and/or type; 2) may be operatively interconnected with the powerhead 12 in any appropriate manner; 3) may be disposed at any appropriate location; 4) may be configured to provide any of the following functions: controlling one or more aspects of the operation of the power injector 10; inputting/editing one or more parameters associated with the operation of the power injector 10; and displaying appropriate information (e.g., associated with the operation of the power injector 10); or 5) any combination of the foregoing. Any appropriate number of GUIs 11 may be utilized. In one embodiment, the power injector 10 includes a GUI 11 that is incorporated by a console that is separate from but which communicates with the powerhead 12. In another embodiment, the power injector 10 includes a GUI 11 that is part of the powerhead 12. In yet another embodiment, the power injector 10 utilizes one GUI 11 on a separate console that communicates with the powerhead 12, and also utilizes another GUI 11 that is on the powerhead 12. Each GUI 11 could provide the same functionality or set of functionalities, or the GUIs 11 may differ in at least some respect in relation to their respective functionalities.

A syringe 28 may be installed on the powerhead 12 and, when installed, may be considered to be part of the power injector 10. Some injection procedures may result in a relatively high pressure being generated within the syringe 28. In this regard, it may be desirable to dispose the syringe 28 within a pressure jacket 26. The pressure jacket 26 is typically associated with the powerhead 12 in a manner that allows the syringe 28 to be disposed therein as a part of or after installing the syringe 28 on the powerhead 12. The same pressure jacket 26 will typically remain associated with the powerhead 12, as various syringes 28 are positioned within and removed from the pressure jacket 26 for multiple injection procedures. The power injector 10 may eliminate the pressure jacket 26 if the power injector 10 is configured/utilized for low-pressure injections and/or if the syringe(s) 28 to be utilized with the power injector 10 is (are) of sufficient durability to withstand high-pressure injections without the additional support provided by a pressure jacket 26. In any case, fluid discharged from the syringe 28 may be directed into a conduit 38 of any appropriate size, shape, configuration, and/or type, which may be fluidly interconnected with the syringe 28 in any appropriate manner, and which may direct fluid to any appropriate location (e.g., to a patient).

The powerhead 12 includes a syringe plunger drive assembly or syringe plunger driver 14 that interacts (e.g., interfaces) with the syringe 28 (e.g., a plunger 32 thereof) to discharge fluid from the syringe 28. This syringe plunger drive assembly 14 includes a drive source 16 (e.g., a motor of any appropriate size, shape, configuration, and/or type, optional gearing, and the like) that powers a drive output 18 (e.g., a rotatable drive screw). A ram 20 may be advanced along an appropriate path (e.g., axial) by the drive output 18. The ram 20 may include a coupler 22 for interacting or interfacing with a corresponding portion of the syringe 28 in a manner that will be discussed below.

The syringe 28 includes a plunger or piston 32 that is movably disposed within a syringe barrel 30 (e.g., for axial reciprocation along an axis coinciding with the double-headed arrow B). The plunger 32 may include a coupler 34. This syringe plunger coupler 34 may interact or interface with the ram coupler 22 to allow the syringe plunger drive assembly 14 to retract the syringe plunger 32 within the syringe barrel 30. The syringe plunger coupler 34 may be in the form of a shaft 36a that extends from a body of the syringe plunger 32, together with a head or button 36b. However, the syringe plunger coupler 34 may be of any appropriate size, shape, configuration, and/or type.

Generally, the syringe plunger drive assembly 14 of the power injector 10 may interact with the syringe plunger 32 of the syringe 28 in any appropriate manner (e.g., by mechanical contact; by an appropriate coupling (mechanical or otherwise)) so as to be able to move or advance the syringe plunger 32 (relative to the syringe barrel 30) in at least one direction (e.g., to discharge fluid from the corresponding syringe 28). That is, although the syringe plunger drive assembly 14 may be capable of bi-directional motion (e.g., via operation of the same drive source 16), the power injector 10 may be configured such that the operation of the syringe plunger drive assembly 14 actually only moves each syringe plunger 32 being used by the power injector 10 in only one direction. However, the syringe plunger drive assembly 14 may be configured to interact with each syringe plunger 32 being used by the power injector 10 so as to be able to move each such syringe plunger 32 in each of two different directions (e.g. in different directions along a common axial path).

Retraction of the syringe plunger 32 may be utilized to accommodate a loading of fluid into the syringe barrel 30 for a subsequent injection or discharge, may be utilized to actually draw fluid into the syringe barrel 30 for a subsequent injection or discharge, or for any other appropriate purpose. Certain configurations may not require that the syringe plunger drive assembly 14 be able to retract the syringe plunger 32, in which case the ram coupler 22 and syringe plunger coupler 34 may not be desired. In this case, the syringe plunger drive assembly 14 may be retracted for purposes of executing another fluid delivery operation (e.g., after another pre-filled syringe 28 has been installed). Even when a ram coupler 22 and syringe plunger coupler 34 are utilized, these components may or may not be coupled when the ram 20 advances the syringe plunger 32 to discharge fluid from the syringe 28 (e.g., the ram 20 may simply “push on” the syringe plunger coupler 34 or directly on a proximal end of the syringe plunger 32). Any single motion or combination of motions in any appropriate dimension or combination of dimensions may be utilized to dispose the ram coupler 22 and syringe plunger coupler 34 in a coupled state or condition, to dispose the ram coupler 22 and syringe plunger coupler 34 in an un-coupled state or condition, or both.

The syringe 28 may be installed on the powerhead 12 in any appropriate manner. For instance, the syringe 28 could be configured to be installed directly on the powerhead 12. In the illustrated embodiment, a housing 24 is appropriately mounted on the powerhead 12 to provide an interface between the syringe 28 and the powerhead 12. This housing 24 may be in the form of an adapter to which one or more configurations of syringes 28 may be installed, and where at least one configuration for a syringe 28 could be installed directly on the powerhead 12 without using any such adapter. The housing 24 may also be in the form of a faceplate to which one or more configurations of syringes 28 may be installed. In this case, it may be such that a faceplate is required to install a syringe 28 on the powerhead 12—the syringe 28 could not be installed on the powerhead 12 without the faceplate. When a pressure jacket 26 is being used, it may be installed on the powerhead 12 in the various manners discussed herein in relation to the syringe 28, and the syringe 28 will then thereafter be installed in the pressure jacket 26.

The housing 24 may be mounted on and remain in a fixed position relative to the powerhead 12 when installing a syringe 28. Another option is to movably interconnect the housing 24 and the powerhead 12 to accommodate installing a syringe 28. For instance, the housing 24 may move within a plane that contains the double-headed arrow A to provide one or more of coupled state or condition and an un-coupled state or condition between the ram coupler 22 and the syringe plunger coupler 34.

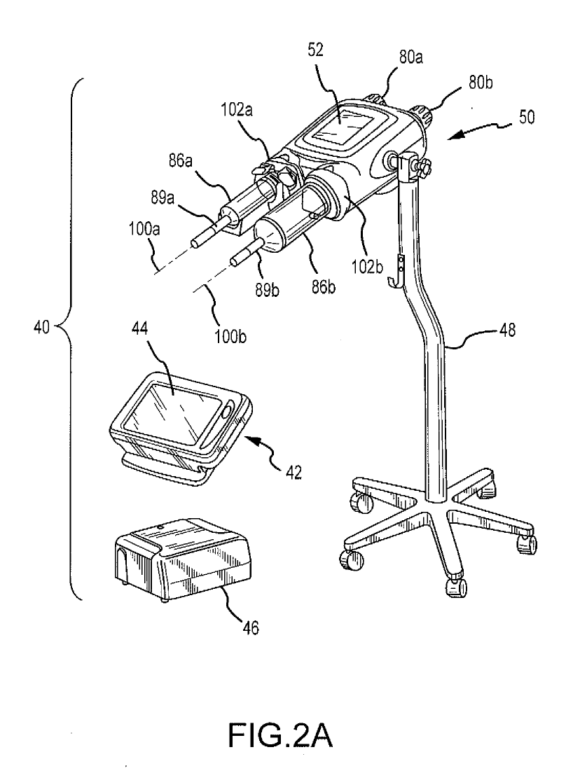

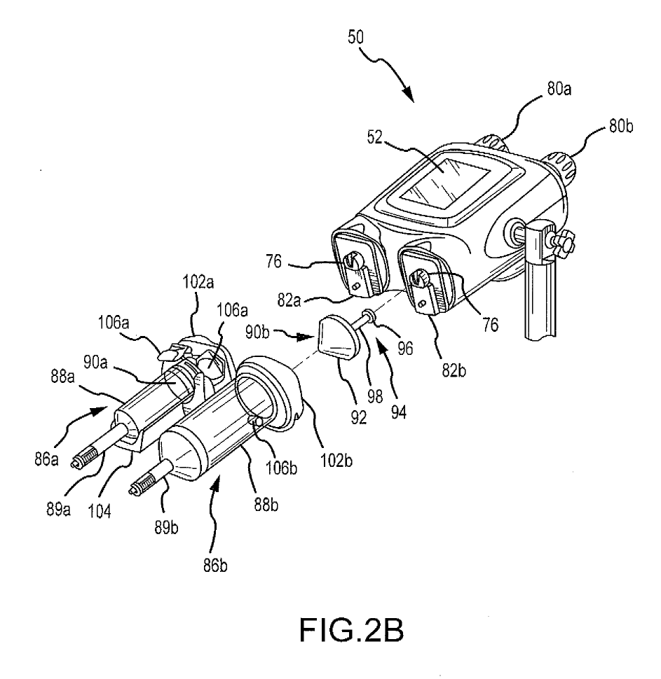

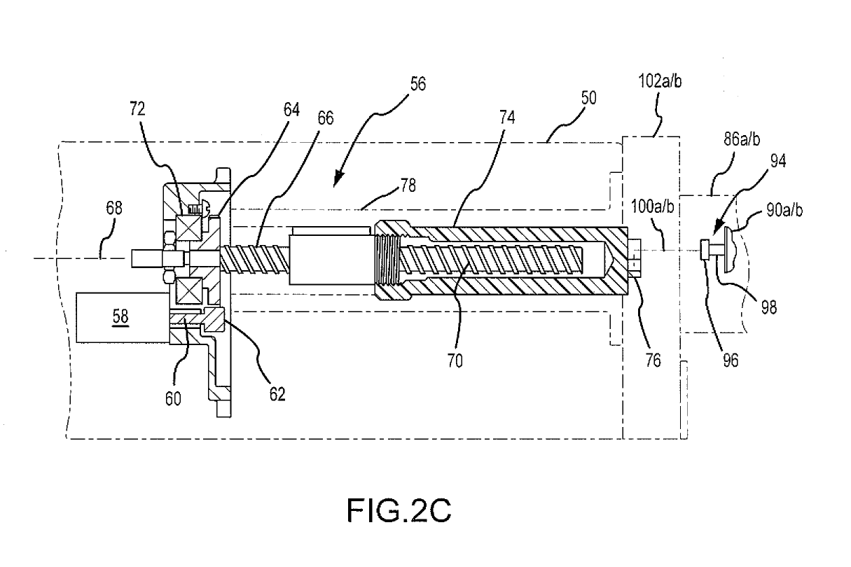

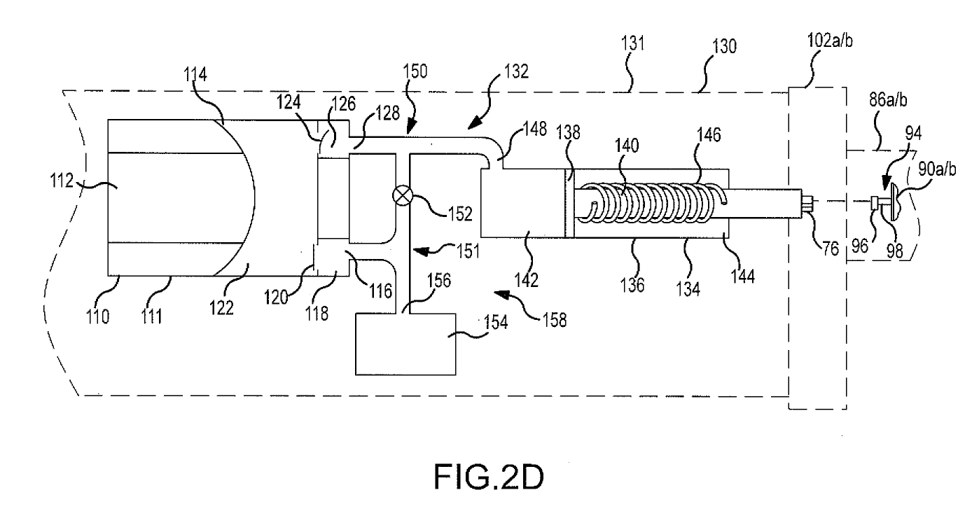

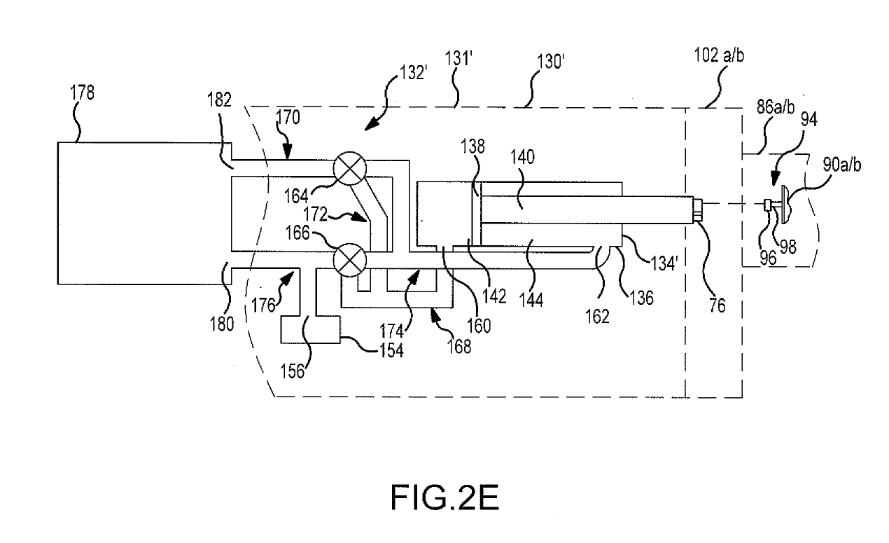

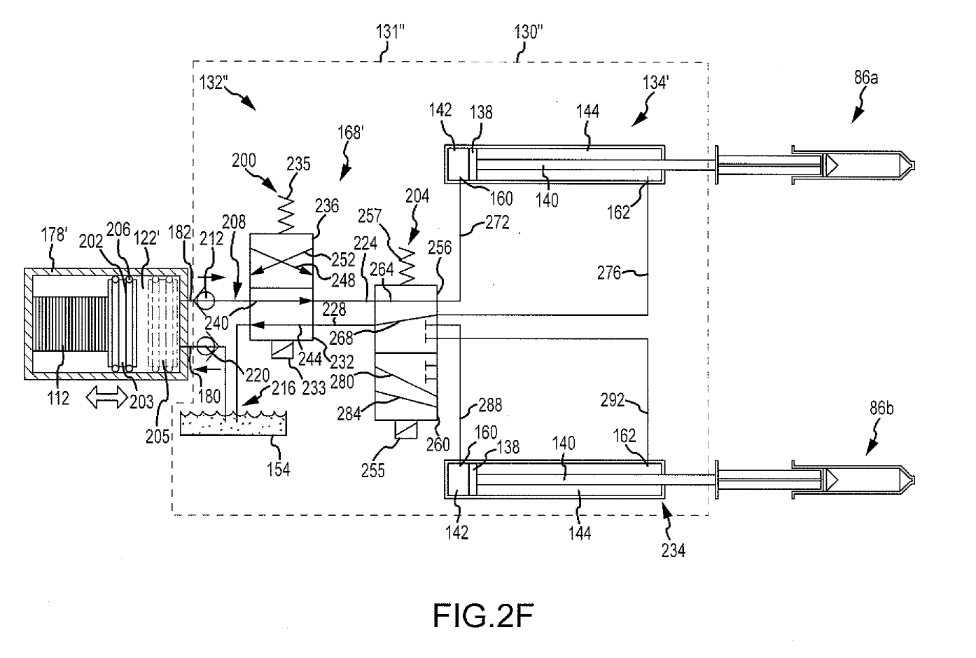

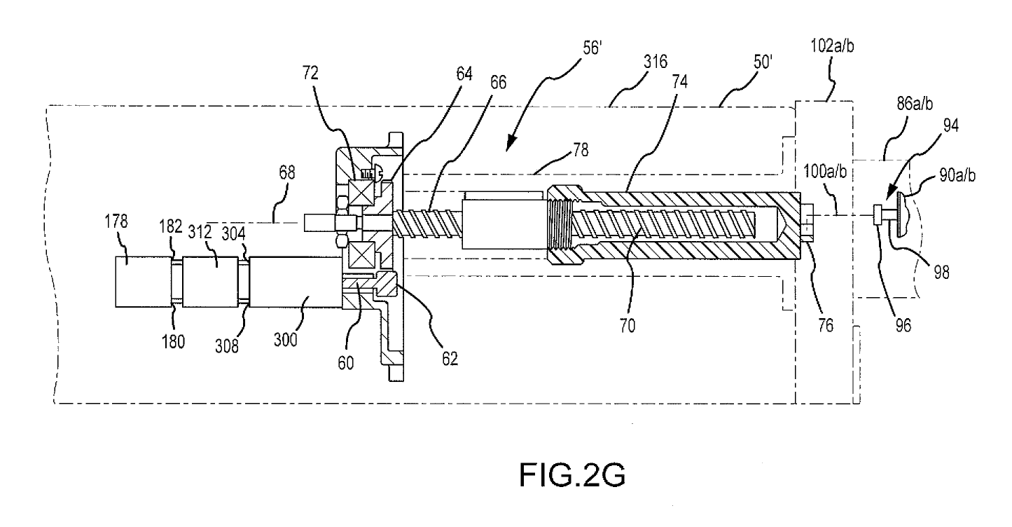

One particular power injector configuration is illustrated in FIG. 2A, is identified by a reference numeral 40, and is at least generally in accordance with the power injector 10 of FIG. 1. The power injector 40 includes a powerhead 50 that is mounted on a portable stand 48. Two syringes 86a, 86b for the power injector 40 are mounted on the powerhead 50. Fluid may be discharged from the syringes 86a, 86b during operation of the power injector 40.

The portable stand 48 may be of any appropriate size, shape, configuration, and/or type. Wheels, rollers, casters, or the like may be utilized to make the stand 48 portable. The powerhead 50 could be maintained in a fixed position relative to the portable stand 48. However, it may be desirable to allow the position of the powerhead 50 to be adjustable relative to the portable stand 48 in at least some manner. For instance, it may be desirable to have the powerhead 50 in one position relative to the portable stand 48 when loading fluid into one or more of the syringes 86a, 86b, and to have the powerhead 50 in a different position relative to the portable stand 48 for performance of an injection procedure. In this regard, the powerhead 50 may be movably interconnected with the portable stand 48 in any appropriate manner (e.g., such that the powerhead 50 may be pivoted through at least a certain range of motion, and thereafter maintained in the desired position).

It should be appreciated that the powerhead 50 could be supported in any appropriate manner for providing fluid. For instance, instead of being mounted on a portable structure, the powerhead 50 could be interconnected with a support assembly, that in turn is mounted to an appropriate structure (e.g., ceiling, wall, floor). Any support assembly for the powerhead 50 may be positionally adjustable in at least some respect (e.g., by having one or more support sections that may be repositioned relative to one or more other support sections), or may be maintained in a fixed position. Moreover, the powerhead 50 may be integrated with any such support assembly so as to either be maintained in a fixed position or so as to be adjustable relative the support assembly.

The powerhead 50 includes a graphical user interface or GUI 52. This GUI 52 may be configured to provide one or any combination of the following functions: controlling one or more aspects of the operation of the power injector 40; inputting/editing one or more parameters associated with the operation of the power injector 40; and displaying appropriate information (e.g., associated with the operation of the power injector 40). The power injector 40 may also include a console 42 and powerpack 46 that each may be in communication with the powerhead 50 in any appropriate manner (e.g., via one or more cables), that may be placed on a table or mounted on an electronics rack in an examination room or at any other appropriate location, or both. The powerpack 46 may include one or more of the following and in any appropriate combination: a power supply for the injector 40; interface circuitry for providing communication between the console 42 and powerhead 50; circuitry for permitting connection of the power injector 40 to remote units such as remote consoles, remote hand or foot control switches, or other original equipment manufacturer (OEM) remote control connections (e.g., to allow for the operation of power injector 40 to be synchronized with the x-ray exposure of an imaging system); and any other appropriate componentry. The console 42 may include a touch screen display 44, which in turn may provide one or more of the following functions and in any appropriate combination: allowing an operator to remotely control one or more aspects of the operation of the power injector 40; allowing an operator to enter/edit one or more parameters associated with the operation of the power injector 40; allowing an operator to specify and store programs for automated operation of the power injector 40 (which can later be automatically executed by the power injector 40 upon initiation by the operator); and displaying any appropriate information relation to the power injector 40 and including any aspect of its operation.

Various details regarding the integration of the syringes 86a, 86b with the powerhead 50 are presented in FIG. 2B. Each of the syringes 86a, 86b includes the same general components. The syringe 86a includes plunger or piston 90a that is movably disposed within a syringe barrel 88a. Movement of the plunger 90a along an axis 100a (FIG. 2A) via operation of the powerhead 50 will discharge fluid from within a syringe barrel 88a through a nozzle 89a of the syringe 86a. An appropriate conduit (not shown) will typically be fluidly interconnected with the nozzle 89a in any appropriate manner to direct fluid to a desired location (e.g., a patient). Similarly, the syringe 86b includes plunger or piston 90b that is movably disposed within a syringe barrel 88b. Movement of the plunger 90b along an axis 100b (FIG. 2A) via operation of the powerhead 50 will discharge fluid from within the syringe barrel 88b through a nozzle 89b of the syringe 86b. An appropriate conduit (not shown) will typically be fluidly interconnected with the nozzle 89b in any appropriate manner to direct fluid to a desired location (e.g., a patient).

The syringe 86a is interconnected with the powerhead 50 via an intermediate faceplate 102a. This faceplate 102a includes a cradle 104 that supports at least part of the syringe barrel 88a, and which may provide/accommodate any additional functionality or combination of functionalities. A mounting 82a is disposed on and is fixed relative to the powerhead 50 for interfacing with the faceplate 102a. A ram coupler 76 of a ram 74 (FIG. 2C), which are each part of a syringe plunger drive assembly or syringe plunger driver 56 (FIG. 2C) for the syringe 86a, is positioned in proximity to the faceplate 102a when mounted on the powerhead 50. Details regarding the syringe plunger drive assembly 56 and variations thereof will be discussed in more detail below in relation to FIGS. 2C-2F. Generally, the ram coupler 76 may be coupled with the syringe plunger 90a of the syringe 86a, and the ram coupler 76 and ram 74 (FIG. 2C) may then be moved relative to the powerhead 50 to move the syringe plunger 90a along the axis 100a (FIG. 2A). It may be such that the ram coupler 76 is engaged with, but not actually coupled to, the syringe plunger 90a when moving the syringe plunger 90a to discharge fluid through the nozzle 89a of the syringe 86a.

The faceplate 102a may be moved at least generally within a plane that is orthogonal to the axes 100a, 100b (associated with movement of the syringe plungers 90a, 90b, respectively, and illustrated in FIG. 2A), both to mount the faceplate 102a on and remove the faceplate 102a from its mounting 82a on the powerhead 50. The faceplate 102a may be used to couple the syringe plunger 90a with its corresponding ram coupler 76 on the powerhead 50. In this regard, the faceplate 102a includes a pair of handles 106a. Generally and with the syringe 86a being initially positioned within the faceplate 102a, the handles 106a may be moved to in turn move/translate the syringe 86a at least generally within a plane that is orthogonal to the axes 100a, 100b (associated with movement of the syringe plungers 90a, 90b, respectively, and illustrated in FIG. 2A). Moving the handles 106a to one position moves/translates the syringe 86a (relative to the faceplate 102a) in an at least generally downward direction to couple its syringe plunger 90a with its corresponding ram coupler 76. Moving the handles 106a to another position moves/translates the syringe 86a (relative to the faceplate 102a) in an at least generally upward direction to uncouple its syringe plunger 90a from its corresponding ram coupler 76.

The syringe 86b is interconnected with the powerhead 50 via an intermediate faceplate 102b. A mounting 82b is disposed on and is fixed relative to the powerhead 50 for interfacing with the faceplate 102b. A ram coupler 76 of a ram 74 (FIG. 2C), which are each part of a syringe plunger drive assembly 56 for the syringe 86b, is positioned in proximity to the faceplate 102b when mounted to the powerhead 50. Details regarding the syringe plunger drive assembly 56 again will be discussed in more detail below in relation to FIG. 2C. Generally, the ram coupler 76 may be coupled with the syringe plunger 90b of the syringe 86b, and the ram coupler 76 and ram 74 (FIG. 2C) may be moved relative to the powerhead 50 to move the syringe plunger 90b along the axis 100b (FIG. 2A). It may be such that the ram coupler 76 is engaged with, but not actually coupled to, the syringe plunger 90b when moving the syringe plunger 90b to discharge fluid through the nozzle 89b of the syringe 86b.

The faceplate 102b may be moved at least generally within a plane that is orthogonal to the axes 100a, 100b (associated with movement of the syringe plungers 90a, 90b, respectively, and illustrated in FIG. 2A), both to mount the faceplate 102b on and remove the faceplate 102b from its mounting 82b on the powerhead 50. The faceplate 102b also may be used to couple the syringe plunger 90b with its corresponding ram coupler 76 on the powerhead 50. In this regard, the faceplate 102b may include a handle 106b. Generally and with the syringe 86b being initially positioned within the faceplate 102b, the syringe 86b may be rotated along its long axis 100b (FIG. 2A) and relative to the faceplate 102b. This rotation may be realized by moving the handle 106b, by grasping and turning the syringe 86b, or both. In any case, this rotation moves/translates both the syringe 86b and the faceplate 102b at least generally within a plane that is orthogonal to the axes 100a, 100b (associated with movement of the syringe plungers 90a, 90b, respectively, and illustrated in FIG. 2A). Rotating the syringe 86b in one direction moves/translates the syringe 86b and faceplate 102b in an at least generally downward direction to couple the syringe plunger 90b with its corresponding ram coupler 76. Rotating the syringe 86b in the opposite direction moves/translates the syringe 86b and faceplate 102b in an at least generally upward direction to uncouple its syringe plunger 90b from its corresponding ram coupler 76.

As illustrated in FIG. 2B, the syringe plunger 90b includes a plunger body 92 and a syringe plunger coupler 94. This syringe plunger coupler 94 includes a shaft 98 that extends from the plunger body 92, along with a head 96 that is spaced from the plunger body 92. Each of the ram couplers 76 includes a larger slot that is positioned behind a smaller slot on the face of the ram coupler 76. The head 96 of the syringe plunger coupler 94 may be positioned within the larger slot of the ram coupler 76, and the shaft 98 of the syringe plunger coupler 94 may extend through the smaller slot on the face of the ram coupler 76 when the syringe plunger 90b and its corresponding ram coupler 76 are in a coupled state or condition. The syringe plunger 90a may include a similar syringe plunger coupler 94 for interfacing with its corresponding ram coupler 76.

The powerhead 50 is utilized to discharge fluid from the syringes 86a, 86b in the case of the power injector 40. That is, the powerhead 50 provides the motive force to discharge fluid from each of the syringes 86a, 86b. One embodiment of what may be characterized as a syringe plunger drive assembly or syringe plunger driver is illustrated in FIG. 2C, is identified by reference numeral 56, and may be utilized by the powerhead 50 to discharge fluid from each of the syringes 86a, 86b. A separate syringe plunger drive assembly 56 may be incorporated into the powerhead 50 for each of the syringes 86a, 86b. In this regard and referring back to FIGS. 2A-B, the powerhead 50 may include hand-operated knobs 80a and 80b for use in separately controlling each of the syringe plunger drive assemblies 56.

Initially and in relation to the syringe plunger drive assembly 56 of FIG. 2C, each of its individual components may be of any appropriate size, shape, configuration and/or type. The syringe plunger drive assembly 56 includes a motor 58, which has an output shaft 60. A drive gear 62 is mounted on and rotates with the output shaft 60 of the motor 58. The drive gear 62 is engaged or is at least engageable with a driven gear 64. This driven gear 64 is mounted on and rotates with a drive screw or shaft 66. The axis about which the drive screw 66 rotates is identified by reference numeral 68. One or more bearings 72 appropriately support the drive screw 66.

A carriage or ram 74 is movably mounted on the drive screw 66. Generally, rotation of the drive screw 66 in one direction axially advances the ram 74 along the drive screw 66 (and thereby along axis 68) in the direction of the corresponding syringe 86a/b, while rotation of the drive screw 66 in the opposite direction axially advances the ram 74 along the drive screw 66 (and thereby along axis 68) away from the corresponding syringe 86a/b. In this regard, the perimeter of at least part of the drive screw 66 includes helical threads 70 that interface with at least part of the ram 74. The ram 74 is also movably mounted within an appropriate bushing 78 that does not allow the ram 74 to rotate during a rotation of the drive screw 66. Therefore, the rotation of the drive screw 66 provides for an axial movement of the ram 74 in a direction determined by the rotational direction of the drive screw 66.

The ram 74 includes a coupler 76 that that may be detachably coupled with a syringe plunger coupler 94 of the syringe plunger 90a/b of the corresponding syringe 86a/b. When the ram coupler 76 and syringe plunger coupler 94 are appropriately coupled, the syringe plunger 90a/b moves along with ram 74. FIG. 2C illustrates a configuration where the syringe 86a/b may be moved along its corresponding axis 100a/b without being coupled to the ram 74. When the syringe 86a/b is moved along its corresponding axis 100a/b such that the head 96 of its syringe plunger 90a/b is aligned with the ram coupler 76, but with the axes 68 still in the offset configuration of FIG. 2C, the syringe 86a/b may be translated within a plane that is orthogonal to the axis 68 along which the ram 74 moves. This establishes a coupled engagement between the ram coupler 76 and the syringe plunger coupler 96 in the above-noted manner.

The power injectors 10, 40 of FIGS. 1 and 2A-C each may be used for any appropriate application, including without limitation for medical imaging applications where fluid is injected into a subject (e.g., a patient). Representative medical imaging applications for the power injectors 10, 40 include without limitation computed tomography or CT imaging, magnetic resonance imaging or MRI, single photon emission computed tomography or SPECT imaging, positron emission tomography or PET imaging, X-ray imaging, angiographic imaging, optical imaging, and ultrasound imaging. The power injectors 10, 40 each could be used alone or in combination with one or more other components. The power injectors 10, 40 each may be operatively interconnected with one or more components, for instance so that information may be conveyed between the power injector 10, 40 and one or more other components (e.g., scan delay information, injection start signal, injection rate).

Any number of syringes may be utilized by each of the power injectors 10, 40, including without limitation single-head configurations (for a single syringe) and dual-head configurations (for two syringes). In the case of a multiple syringe configuration, each power injector 10, 40 may discharge fluid from the various syringes in any appropriate manner and according to any timing sequence (e.g., sequential discharges from two or more syringes, simultaneous discharges from two or more syringes, or any combination thereof). Multiple syringes may discharge into a common conduit (e.g., for provision to a single injection site), or one syringe may discharge into one conduit (e.g., for provision to one injection site), while another syringe may discharge into a different conduit (e.g., for provision to a different injection site). Each such syringe utilized by each of the power injectors 10, 40 may include any appropriate fluid (e.g., a medical fluid), for instance contrast media, a radiopharmaceutical, saline, and any combination thereof. Each such syringe utilized by each of the power injectors 10, 40 may be installed in any appropriate manner (e.g., rear-loading configurations may be utilized; front-loading configurations may be utilized; side-loading configurations may be utilized).

FIG. 2D presents a variation of the powerhead 50 of FIG. 2C in the form of a powerhead 130. Corresponding components between the two embodiments are identified by common reference numerals, and each of the individual components of the powerhead 130 may be of any appropriate size, shape, configuration and/or type. The powerhead 130 may include a housing 131 having a syringe plunger drive assembly 132 mounted therein to discharge fluid from each of the syringes 86a, 86b. The syringe plunger drive assembly 132 may broadly include a syringe plunger driver 134, a piezoelectric pump 110 and an interconnecting arrangement 158 that fluidly interconnects the syringe plunger driver 134 to the piezoelectric pump 110 and allows the piezoelectric pump 110 to supply pressurized hydraulic fluid (not shown) to the syringe plunger driver 134. Any appropriate hydraulic fluid may be utilized (e.g., oil).

The syringe plunger drive assembly 132 may be constructed of all nonferrous components. The syringe plunger driver 134 may be in the form of a single-acting hydraulic cylinder that includes a piston 138 and a piston rod 140. The piston rod 140 may be appropriately attached to the piston 138, and together the piston 138 and piston rod 140 collectively reciprocate in two opposed directions (e.g., a forward or first direction (toward the right in FIG. 2D)) and a rearward or second direction (toward the left in FIG. 2D)) within a body 136 of the syringe plunger driver 134. The piston rod 140 may be interconnected to the coupler 76 for detachably coupling with the syringe plunger coupler 94 of the syringe plunger 90a/b of the corresponding syringe 86a/b. The piston 138 divides the body 136 into a first chamber 142 for containing hydraulic fluid and a second chamber 144. The second chamber 144 may contain another fluid (e.g., air). The piston 138 may be designed to reduce the potential of hydraulic fluid flow between the first and second chambers 142, 144.

A spring 146, which may be in the form of any appropriate biasing member, may be wrapped around the piston rod 140 within the second chamber 144 to provide a return force against the piston 138 to assist in moving the piston 138 and piston rod 140 in at least the rearward direction. The spring 146 may be interconnected in any appropriate way to the piston 138 and a portion of the second chamber 144 (e.g., an end cap of the syringe plunger driver 134). While spring 146 is illustrated as being wrapped around the piston rod 140 in the second chamber 144, it is contemplated that the spring 146 could be appropriately mounted within the first chamber 142 to provide a pulling return force instead of a pushing return force as is presently illustrated in FIG. 2E. Other types of biasing members/elements may be utilized. Any appropriate number of biasing elements may be utilized, and furthermore may be incorporated in any appropriate manner.

The body 136 additionally includes at least one port 148 for allowing pressurized hydraulic fluid from the piezoelectric pump 110 to enter the first chamber 142 and drive the piston 138 and the piston rod 140 in the forward direction and against the return force of the spring 146. Additionally, once a valve 152 of the interconnecting arrangement 158 is open, the return force provided by the spring 146 may cause hydraulic fluid to exit the first chamber 142 through port 148 and flow into a reservoir 154; the piston 138 and piston rod 140 can concurrently move in the rearward direction as will be more fully described below.

In an exemplary embodiment, the piezoelectric pump 110 may be composed generally or entirely of non-ferrous components and includes a body 111 with a piezoelectric stack 112 and a diaphragm 114 within the body 111 to pump a hydraulic fluid (e.g., oil) as will be described below. While one type of non-ferrous piezoelectric pump 110 will be described with reference to FIG. 2D, other appropriate types of non-ferrous piezoelectric pumps may be used. The diaphragm 114 generally defines an adjustable border of a pumping chamber 122 within the body 111. To allow the piezoelectric pump 110 to be fluidly interconnected with other components (e.g., the syringe plunger driver 134), the piezoelectric pump 110 includes an inlet port 116 and an outlet port 128. While not specifically shown in FIG. 20, inlet port 116 and outlet port 128 may include or be in the form of any appropriate type of connector or joint known in the art to allow the piezoelectric pump 110 to be fluidly interconnected to any appropriate type or types of components.

An inlet chamber 118 and an inlet valve 120 may fluidly interconnect the inlet port 116 to the pumping chamber 122. Similarly, an outlet chamber 126 and an outlet valve 124 may fluidly interconnect the outlet port 128 to the pumping chamber 122. The inlet valve 120 and the outlet valve 124 each may be employed in the form of a one-way flow valve, such as a reed valve. The inlet and outlet valves 120, 124 may also be formed as any other appropriate types of valves. The diaphragm 114 may be made of a metallic or other material and may be preloaded when appropriately installed adjacent to the piezoelectric stack 112.

The piezoelectric stack 112 may be made of any appropriate piezoelectric material such as lead zirconate titanate (PZT), various crystals and ceramics, and the like. Further, the piezoelectric stack 112 is appropriately connected to a power supply (not shown). When an electrical voltage is applied to the piezoelectric stack 112, the piezoelectric stack 112 may either contract or expand. As illustrated in FIG. 2D, when a positive electrical voltage has been applied to the piezoelectric stack 112, the piezoelectric stack 112 may expand so as to deflect the diaphragm 114 towards the inlet and outlet ports 116, 128, and in turn the pressure of fluid within the pumping chamber 122 increases relative to the fluid within the outlet chamber 126 and the inlet chamber 118. As a result and as shown in FIG. 2D, inlet valve 120 closes, outlet valve 124 opens, and fluid flows out of outlet port 128 and into the syringe plunger driver 134 to provide power to move the piston 138 in a forward direction. In contrast, if no electric voltage, a negative electric voltage or at least a different electrical voltage is applied to the piezoelectric stack 112, the piezoelectric stack 112 may contract (relative to the position illustrated in FIG. 2D) so as to deflect the diaphragm 114 away from the inlet and outlet ports 116, 128, thus decreasing the pressure of fluid within the pumping chamber 122 relative to fluid within outlet chamber 126 and the inlet chamber 118. Consequently, outlet valve 124 closes, inlet valve 120 opens, and fluid flows into inlet port 116 and through inlet chamber 118 and inlet valve 120 into the pumping chamber 122 to provide fluid for the expansion cycle of the piezoelectric stack 112 (the configuration where valve 124 is closed and valve 120 is open is not shown in FIG. 20). By controlling the voltage level to the piezoelectric stack 112, motion of the diaphragm 114 may be accurately controlled, achieving the desired levels of fluid flow rate and pressure.

The outlet port 128 may be fluidly interconnected to the port 148 of the syringe plunger driver 134 and a valve 152 by a first interconnecting assembly 150. Additionally, inlet port 116 may be fluidly interconnected to the port 156 of a reservoir 154 and the valve 152 by a second interconnecting assembly 151. As such, each of the valve 152 and the reservoir 154 is fluidly interconnected or interconnectable to each of the piezoelectric pump 110 and the syringe plunger driver 134. First and second interconnecting assemblies 150, 151 may comprise any combination, arrangement or connection of various appropriate types of tubes, pipes, and the like and/or may be formed by any appropriate process such as by extruding, a lost foam process, and the like. The valve 152 may be in the form of an electrically controlled valve moveable between a closed position (e.g., where no flow can occur through the valve 152) and various open positions. The various open positions may range from a fully open position to other positions where the flow between the first interconnecting assembly 150 and the second interconnecting assembly 151 is selectively throttled (e.g., the flow rate between first interconnecting assembly 150 and the second interconnecting assembly 151 may be controlled). While the valve 152 may be in the form of a non-ferrous piezoelectric valve, other appropriate types of non-ferrous valve arrangements may also be used.

Combined operation of the piezoelectric pump 110, valve 152, and syringe plunger driver 134 to produce a forward stroke of the syringe plunger driver 134 will now be described. When piezoelectric stack 112 expands, thus deflecting diaphragm 114 toward inlet and outlet ports 120, 124, hydraulic fluid pressure within pumping chamber 122 may increase relative to that in the outlet chamber 126. As a result, outlet valve 124 may open and pressurized hydraulic fluid may flow through the first interconnecting assembly 150 and into the first chamber 142 to drive the piston 138 and the piston rod 140 generally in a forward direction against the return force of the spring 146 and any resistance from the syringe plungers 90 a/b of the syringes 86a/b. It is also noted that during the forward stroke of the piston rod 140, the valve 152 is closed, thus reducing the potential of hydraulic fluid traveling from the first interconnecting assembly 150 to the second interconnecting assembly 151. Due to the relative incompressibility of the hydraulic fluid used in the syringe plunger drive assembly 132, expansion of piezoelectric stack 112 and a corresponding deflection of the diaphragm 114 causes a proportional forward movement of the piston 138 and the piston rod 140 resulting in a controlled forward movement of the syringe plunger driver 134. If a single incremental expansion of the piezoelectric stack 112 does not produce a desired magnitude of a forward movement of the piston 138 and piston rod 140, then the piezoelectric stack 112 may contract and then expand again to produce another incremental forward movement of the piston 138 and piston rod 140. When the piezoelectric stack 112 contracts, a negative pressure region will be created in the pumping chamber 122 relative to the pressure in the second interconnecting assembly 151 and the reservoir 154, and the inlet port 120 will open, allowing fluid to enter the pumping chamber 122 and thereafter be available for a subsequent expansion of the piezoelectric stack 112. Of course, it may readily be determined how many expansion/contraction cycles of the piezoelectric stack 112 are required to produce a desired magnitude of a forward movement of the piston 138 and piston rod 140.

To move the piston 138 and piston rod 140 in the rearward direction, the valve 152 may be moved to an open position. As noted, the spring 146 urges the piston 138 and piston rod 140 in the rearward direction. Because the valve 152 is now open and the first chamber 142 is fluidly connected to the reservoir 154, fluid may flow from the first chamber 142, through the first interconnecting assembly 150, valve 152, and second interconnecting assembly 151, and into the reservoir 154. in this regard, the pressure within the reservoir 154 may be selected and/or controlled such that it is lower than the pressure within the first chamber 142 (due to the action of the spring 146) for any piston 138 position thus allowing the spring 146 to move the piston 138 to its most rearward position. It is noted that the rearward or return stroke of the piston 138 and piston rod 140 may be controlled by throttling the valve 152 or otherwise controlling the degree to which the valve 152 allows hydraulic fluid to freely flow from the first interconnecting assembly 151 to the second interconnecting assembly 152.

If the second chamber 144 is not open to atmospheric pressure, it may include a check valve or other arrangement (not shown) to allow for the expelling of fluid (e.g., air) if the pressure of the fluid within the second chamber 144 becomes too great during a forward stroke of the piston 138 and piston rod 140. Such a check valve or other arrangement could also allow for the introduction of fluid (e.g., air) into the second chamber 144 during a rearward stroke of the piston 138 and piston rod 140 (e.g., to prevent the formation of a vacuum).

FIG. 2E presents a variation of the powerhead 130 of FIG. 2D and corresponding components between the embodiments are identified by common reference numerals. This embodiment includes a powerhead 130′ which may be of any appropriate size, shape, configuration and/or type. Those corresponding components that differ in at least some respect from the embodiment of FIG. 2D are identified by a “single prime” designation in FIG. 2E. The powerhead 130′ includes a housing 131′ having a syringe plunger drive assembly 132′ appropriately mounted within the housing 131′ to discharge fluid from each of the syringes 86a, 86b. The primary differences between the syringe plunger drive assembly 132 of FIG. 2D and the syringe plunger drive assembly 132′ of FIG. 2E are: the use of a double acting hydraulic cylinder as the syringe plunger driver 134′ in place of the single acting hydraulic cylinder of FIG. 2E; and first and second valves 164, 166 allowing for pressurized hydraulic fluid to selectively flow into and out of first and second ports 160, 162 of the syringe plunger driver 134′. The first and second valves 164, 166 are part of an interconnecting arrangement 168 that fluidly interconnects the syringe plunger driver 134′ to a pump 178. The pump 178 may be in the form of various appropriate types of piezoelectric pumps, including the piezoelectric pump 110 of FIG. 2D. As will be described below, such an arrangement allows pressurized hydraulic fluid from the pump 178 to drive both the forward and rearward strokes of the piston 138 and the piston rod 140.

The interconnecting arrangement 168 is generally composed of first, second, third, and fourth interconnecting assemblies 170, 172, 174, 176. However, it will be recognized that any number of interconnecting assemblies may be used. The first interconnecting assembly 170 generally may fluidly interconnect outlet port 182 of the pump 178 to the first valve 164. The second interconnecting assembly 172 generally may fluidly interconnect the first valve 164, second valve 166, and first port 160, while the third interconnecting assembly 174 generally may fluidly interconnect the first valve 164, second valve 166, and second port 162. The fourth interconnecting assembly 176 generally may fluidly interconnect the second valve 166, port 156 of reservoir 154, and inlet port 180 of the pump 178. Each of the first and second valves 164, 166 may be in the form of a three-way valve that allows for hydraulic fluid flow between two of the various interconnecting assemblies 170, 172, 174, 176 in each of various positions. For instance, when first valve 164 is in a first position, hydraulic fluid may flow between the first and second interconnecting assemblies 170, 172, but cannot flow between either of the first and second interconnecting assemblies 170, 172 and the third interconnecting assembly 174. When first valve 164 is in a second position, hydraulic fluid may flow between the first and third interconnecting assemblies 170, 174, but cannot flow between either of the first and third interconnecting assemblies 170, 174 and the second interconnecting assembly 172. Similarly, when second valve 166 is in a first position, hydraulic fluid may flow between the third and fourth interconnecting assemblies 174, 176, but cannot flow between either of the third and fourth interconnecting assemblies 174, 176 and second interconnecting assembly 172. Moreover, when second valve 166 is in a second position, hydraulic fluid may flow between the second and fourth interconnecting assemblies 172, 176, but cannot flow between either of the second and fourth interconnecting assemblies 172, 176 and third interconnecting assembly 174.

In operation, the following interactions generally take place to produce a forward stroke or movement of the piston 138 and the piston rod 140. With each of the first and second valves 164, 166 set to their respective first position, pressurized hydraulic fluid may flow from the outlet port 182 of the pump 178 through the first interconnecting assembly 170, first valve 164 and second interconnecting assembly 172, and into the first chamber 142 to drive the piston 138 and piston rod 140. The forward stroke of the piston 138 and piston rod 140 may be controlled due to the incompressible nature of the hydraulic fluid. As with other embodiments, the pump 178 may draw hydraulic fluid (e.g., during a suction or drawing phase of the pump 178) into the inlet port 180 from the reservoir 154.

At the same time hydraulic fluid is entering the first chamber 142 and driving the piston 138 and piston rod 140, hydraulic fluid may be exiting the second port 162 and entering the third interconnecting assembly 174. While hydraulic fluid cannot flow from the third interconnecting assembly 174 to either of the first or second interconnecting assemblies 170, 172 at this time (due to the position of valve 166), hydraulic fluid may flow from the third interconnecting assembly 174 to the fourth interconnecting assembly 176. Once the hydraulic fluid has entered the fourth interconnecting assembly 176, it may generally flow into the inlet port 180 of the pump 178. Pressure fluctuations in the syringe plunger drive assembly 132′ (e.g., due to the pumping action of the pump 178) may generally be accommodated by the reservoir 154.

In order to provide a driving force for the rearward stroke of the piston 138 and the piston rod 140, each of the first and second valves 164, 166 may be moved to their respective second position. With each of the first and second valves 164, 166 set to their respective second position, pressurized hydraulic fluid may flow from the outlet port 182 of the pump 178 through the first interconnecting assembly 170, first valve 164 and third interconnecting assembly 174, and into the second chamber 144 to drive the piston 138 and piston rod 140 in the rearward direction.

Simultaneous with hydraulic fluid entering the second chamber 144, hydraulic fluid may be exiting the first chamber 142 through first port 160. Once the hydraulic fluid exits the first port 160, it may travel through the second interconnecting assembly 172, through second valve 166, and into the fourth interconnecting assembly 176. Because the first valve 164 is in the second position, fluid cannot travel from the second interconnecting assembly 172 to either the first or third interconnecting assemblies 170, 174. Again, pressure fluctuations in the syringe plunger drive assembly 132′ due to the pumping action of the pump 178 may generally be accommodated by the reservoir 154.

A separate syringe plunger drive assembly 132, 132′ (e.g. piezoelectric pump 110/pump 178, interconnecting arrangement 158, 168 and syringe plunger driver 134, 134′) may be incorporated into the powerhead 130, 130′ of the embodiments of FIGS. 2D and 2E for each of the syringes 86a, 86b. It is also contemplated that each of the syringe plunger drive assemblies 132, 132′ may be driven by the same piezoelectric pump (e.g., pump 110 or pump 178). In such embodiments, each of the interconnecting assemblies 158, 168 would be appropriately modified to allow for pressurized hydraulic fluid to selectively flow to both of the syringe plunger drivers 134, 134′ either individually or simultaneously.

FIG. 2F presents a variation of the powerhead 130′ of FIG. 2E and corresponding components between the embodiments are identified by common reference numerals. Those corresponding components that differ in at least some respect from the embodiment of FIG. 2E are identified by a “double prime” designation. The embodiment of FIG. 2F includes a powerhead 130″ which may be of any appropriate size, shape configuration and/or type. The powerhead 130″ includes a housing 131″ having a syringe plunger drive assembly 132″ appropriately mounted within the housing 131″ to discharge fluid from each of the syringes 86a, 86b. The primary differences between the syringe plunger drive assembly 132′ of FIG. 2E and the syringe plunger drive assembly 132″ of FIG. 2F are: a) the use of a pump 178′ that includes a piston but that does not include inlet and outlet chambers or inlet and outlet valves; b) the use of a second syringe plunger driver 234 that may be used in conjunction with a first syringe plunger driver (e.g., syringe plunger driver 134′), both of which may be driven by the pump 178′; and c) the use of a directional valve 200 and selector valve 204, each of which may be appropriately manipulated to selectively allow pressurized hydraulic fluid to flow to the first or second chamber 142, 144 of the first or second syringe plunger driver 134′, 234 through first and/or second ports 160, 162.

The pump 178′ may include a piston 202 of any appropriate form that may be appropriately connected to the piezoelectric stack 112 and that is operable to seal the hydraulic fluid in the pumping chamber 122′ from other portions of the pump 178′. As illustrated, the piston 202 may include a pair of gaskets 206, each of which may be in the form of an o-ring to provide the previously-mentioned sealing feature, although other sealing devices are contemplated. As with the diaphragm 114 of other embodiments, the piston 202 may move in a first direction to push hydraulic fluid out of the pumping chamber 122′ through the outlet 182 as the piezoelectric stack 112 expands or contracts. The hydraulic fluid generally may not be operable to flow out of the inlet 180 due to a second check valve 220 as will be later described. The piston 202 may move in a second direction to draw hydraulic fluid into the pumping chamber 122′ through the inlet 180 as the piezoelectric stack 112 contracts or expands, and the hydraulic fluid generally may not flow through the outlet 182 because of a first check valve 212 as will be later described. As can be seen in FIG. 2F, the piston 202 may be operable to move between at least first and second positions 203, 205, although the piston 202 may assume or travel between many other positions within the pump 178′.

The directional valve 200 and selector valve 204 are part of an interconnecting arrangement 168′ that fluidly interconnects the syringe plunger drivers 134′, 234 to the pump 178′. Additionally, the interconnecting arrangement 168′ also includes any appropriate number of interconnecting assemblies (e.g., tubes, pipes) that serve to fluidly interconnect the pump 178′, reservoir 154, directional and selector valves 200, 204, and first and second syringe plunger drivers 134′, 234 as will be described below. While one embodiment of the interconnecting arrangement 168′ will be described below, it will be appreciated that any arrangement that allows pressurized hydraulic fluid from the pump 178′ to drive both the forward and rearward strokes of the piston 138 and piston rod 140 of each of the first and second syringe plunger drivers 134′, 234 is contemplated as being within the scope of the embodiments. Further, while at least some of the below discussion will be applicable to the first and second syringe plunger drivers 134′, 234, each being in the form of a double-acting hydraulic cylinder, in other embodiments one or more of the first and second syringe plunger drivers 134′, 234 may be in the form of a single-acting hydraulic cylinder. In such embodiments, the hydraulic fluid would only drive the forward stroke of the one or more single-acting hydraulic cylinders, and the interconnecting arrangement 168′ would be designed accordingly.

The outlet port 182 of the pump 178′ may be appropriately fluidly connected to the directional valve 200 by way of a first interconnecting assembly 208, along with the first check valve 212 that may be appropriately connected in series with the first interconnecting assembly 208. In this regard, hydraulic fluid may be operable to flow within the first interconnecting assembly 208 in a direction from the pump 178′ towards the directional valve 200 but not vice versa. Similarly, the inlet port 180 of the pump 178′ may be appropriately fluidly connected to the directional valve 200 by way of a second interconnecting assembly 216, along with the second check valve 220 and the reservoir 154, each of which may be appropriately connected in series with the second interconnecting assembly 216.

The directional valve 200 may be any appropriate valve that allows fluid from the first interconnecting assembly 208 to selectively flow to either a first or second intermediate interconnecting assembly 224, 228 and fluid from either the first or second intermediate interconnecting assembly 224, 228 to flow to the second interconnecting assembly 208. For instance, the directional valve 200 may be a piezoelectrically-activated directional valve that includes selectively activated first and second sections 232, 236, each of which may respectively correspond to first and second positions of the directional valve 200. A piezoelectric element 233 and a biasing member 235 (which may be in the form of a spring) may assist in shifting or otherwise orienting the directional valve 200 between the first and second positions. The first section 232 may include first and second passages 240, 244 (e.g., tubes, machined conduits), the first passage 240 allowing fluid to flow from the first interconnecting assembly 208 to the first intermediate connecting assembly 224 and the second passage 244 allowing fluid to flow from the second intermediate interconnecting assembly 228 to the second interconnecting assembly 216. The second section 236 may include first and second passages 248, 252 (e.g., tubes, machined conduits), the first passage 248 allowing fluid to flow from the first interconnecting assembly 208 to the second intermediate connecting assembly 228 and the second passage 252 allowing fluid to flow from the first intermediate interconnecting assembly 224 to the second interconnecting assembly 216. As will be described below, the directional valve 200 may be selectively positioned such that fluid can flow through one of the first and second sections 232, 236 by an injection protocol or in another appropriate manner during an injection procedure or the like.

The selector valve 204 may be in the form of any valve allowing fluid to selectively flow between the first intermediate interconnecting assembly 224 and one of the first chambers 142 of the first or second syringe plunger driver 134′, 234, and to selectively flow between the second intermediate interconnecting assembly 228 and one of the second chambers 144 of the first or second syringe plunger driver 134′, 234. As an illustration, the selector valve 204 may be in the form of a piezo-activated selector valve that includes selectively activated first and second sections 256, 260, each of which may respectively correspond to first and second positions of the selector valve 204. A piezoelectric element 255 and a biasing member 257 (which may be in the form of a spring) may assist in shifting or otherwise orienting the selector valve 204 between the first and second positions. The first section 256 may include first and second passages 264, 268: the first passage 264 allows fluid to flow between the first intermediate interconnecting assembly 224, the first chamber interconnecting assembly 272 of the first syringe plunger driver 134′, and the first chamber 142 of the first syringe plunger driver 134′; and the second passage 268 allows fluid to flow between the second intermediate interconnecting assembly 228, the second chamber interconnecting assembly 276 of the first syringe plunger driver 134′, and the second chamber 144 of the first syringe plunger driver 134′. The second section 260 may include first and second passages 280, 284: the first passage 280 allows fluid to flow between the first intermediate interconnecting assembly 224, the first chamber interconnecting assembly 288 of the second syringe plunger driver 234, and the first chamber 142 of the second syringe plunger driver 234; and the second passage 284 allows fluid to flow between the second intermediate interconnecting assembly 228, the second chamber interconnecting assembly 292 of the second syringe plunger driver 234, and the second chamber 144 of the second syringe plunger driver 234. Each of the variously described interconnecting assemblies may be of any appropriate design (e.g., tubes, pipes, conduits) and may be connected to the pump 178′, reservoir 154, directional valve 200, selector valve 204, and first and second syringe plunger drivers 134′, 234 in any appropriate manner.

To produce a forward stroke or movement of the piston 138 and the piston rod 140 of the first syringe plunger driver 134′, each of the directional and selector and valves 200, 204 is set to its respective first position (e.g., as illustrated in FIG. 2F). Pressurized hydraulic fluid may then flow from the outlet port 182 of the pump 178′ through the first check valve 212, first interconnecting assembly 208, first passage 240 of the first section 232 of the directional valve 200, first intermediate interconnecting assembly 224, first passage 264 of the first section 256 of the selector valve 204, and first chamber interconnecting assembly 272 to the first chamber 142 of the first syringe plunger driver 134′ to drive the piston 138 and piston rod 140. As with other embodiments, the forward stroke of the piston 138 and piston rod 140 may be controlled due to the incompressible nature of the hydraulic fluid, and the pump 178′ may draw hydraulic fluid (e.g., during a suction or drawing phase of the pump 178′) into the inlet port 180 from the reservoir 154.

As hydraulic fluid fills the first chamber 142 and drives the piston 138 and piston rod 140, hydraulic fluid exits the second chamber 144 and passes through the second chamber interconnecting assembly 276, second passage 268 of the first section 256 of the selector valve 204, second intermediate interconnecting assembly 228, second passage 244 of the first section 232 of the directional valve 200, and second interconnecting assembly 216 to the reservoir 154. Pressure fluctuations in the syringe plunger drive assembly 132″ (e.g., due to the pumping action of the pump 178′) may generally be accommodated by the reservoir 154.

To provide a driving force for the rearward stroke of the piston 138 and the piston rod 140 of the first syringe plunger driver 134′, the directional valve 200 may be appropriately shifted to or otherwise situated in its second position such that pressurized hydraulic fluid may flow through the second section 236 of the directional valve 200. More specifically, the fluid may flow from the first interconnecting assembly 208 through the first passage 248 of the second section 236 to the second intermediate interconnecting assembly 228, second passage 268 of the first section 256 of the selector valve 204, and second chamber interconnecting assembly 276, and into the second chamber 144 to drive the rearward stroke of the piston 138 and piston rod 140 of the first syringe plunger driver 134′. Fluid exiting the first chamber 142 of the first syringe plunger driver 134′ may travel back through the various interconnecting assemblies, and selector and directional valves 204, 200 to the reservoir 154.

To drive the forward stroke of the second syringe plunger driver 234, the directional valve 200 is appropriately disposed in its first position and the selector valve 204 is appropriately disposed in its second position. Thereafter, pressurized hydraulic fluid may travel from the pump 178′ through the various interconnecting assemblies and valves to the first chamber 142 of the second syringe plunger driver 234. Similarly, once the directional valve 200 is disposed in its second position, the rearward stroke of the second syringe plunger driver 234 can now be carried out by allowing pressurized hydraulic fluid to travel from the pump 178′ through the various interconnecting assemblies and valve to the second chamber 144 of the second syringe plunger driver 234.

While the above described powerhead 130″ has been described as driving each of the first and second syringe plunger drivers 134′, 234 individually, the interconnecting arrangement 168″ may also be designed such that the pump 178′ can supply pressurized hydraulic fluid to the first and second syringe plunger drivers 134′, 234 simultaneously. For instance, alternate or additional valves may be incorporated into the powerhead 130″ that could divert pressurized hydraulic fluid to the first chambers 142 of the first and second syringe plunger drivers 134′, 234, either equally or in differing proportions, so as to simultaneously drive the piston 138 and piston rod 140 of each of the first and second syringe plunger drivers 134′, 234 and correspondingly the plungers of two different syringes. Other arrangements are contemplated.

FIG. 2G presents a variation of the powerhead 50 of FIG. 2C and corresponding components between the embodiments are identified by a common reference numeral. Those corresponding components that differ in at least some respect from the embodiment of FIG. 20 are identified by a “single prime” designation in FIG. 2G. Each of the individual components of a powerhead 50′ of this embodiment may be of any appropriate size, shape, configuration and/or type. The primary difference between the powerhead 50 of FIG. 2C and the powerhead 50′ of FIG. 2G is that the syringe plunger drive assembly 56′ does not use the above described motor 58. Instead, the syringe plunger drive assembly 56′ includes a hydraulic motor 300 powered by the above-described pump 178, and again which uses a piezoelectric drive source.

The hydraulic motor 300 may be constructed of all non-ferrous components and may include a first port 304 and a second port 308 for appropriate fluid connection to the inlet port 180 and outlet port 182 of the pump 178 by an interconnecting arrangement 312. The interconnecting arrangement 312 may include any arrangement of tubing, valves, reservoirs, and the like to allow the pump 178 to selectively supply pressurized hydraulic fluid to either of the first and second ports 304, 308. In an exemplary embodiment, the interconnecting arrangement 312 may be in the form of the interconnecting arrangement 168 of the embodiment illustrated in FIG. 2E. However, other arrangements that allow pressurized hydraulic fluid to flow to either of the first and second ports 304, 308 of the hydraulic motor 300 are contemplated.

Additionally, the hydraulic motor 300 may also be appropriately connected to the output shaft 60 and drive gear 62. As previously discussed, a driving gear 64 may be mounted on a drive screw 66. Thus, rotation of the drive gear 62 in a first or second direction causes simultaneous rotation of the driven gear 64 and the drive screw 66, and ultimately axial advancement of the ram 74 along the drive screw 66 (and thereby along axis 68) either towards or away from the corresponding syringe 86a/b. In this regard, the hydraulic motor 300 assists in rotating the drive screw 66 to move the ram 74 along the drive screw 66 in order to move a corresponding syringe plunger 90a/b of a syringe 86a/b. The hydraulic motor 300 causes the output shaft 60 and drive gear 62 to rotate in a first direction when a pressurized hydraulic fluid is supplied through the first port 304, and in a second direction when the pressurized hydraulic fluid is supplied through the second port 308. Alternatively, the hydraulic motor 300 may be connected directly to the drive screw 66.

While each of the hydraulic motor 300, interconnecting arrangement 312 and pump 178 is shown in FIG. 2G as being located within a housing 316 of the powerhead 50′, it is contemplated that at least one of the pump 178, interconnecting arrangement 312 or hydraulic motor 300 or even other components could be located outside of the housing 316. Additionally, while a single hydraulic motor 300 has been illustrated in FIG. 2G, it is envisioned that a plurality of hydraulic motors 300 (e.g., a pair of hydraulic motors 300) may be used in conjunction with the pump 178. For instance, modifying the interconnecting arrangement 312 to be similar to the interconnecting arrangement 168′ of FIG. 2F would allow pressurized hydraulic fluid to selectively flow between the pump 178 and one or more of the plurality of hydraulic motors 300. Other arrangements contemplate that different types of syringe plunger drivers may be used with the pump 178, 178′. As an example, a double-acting hydraulic cylinder and a hydraulic motor may be utilized in the same powerhead 50′, 130′, 130″.

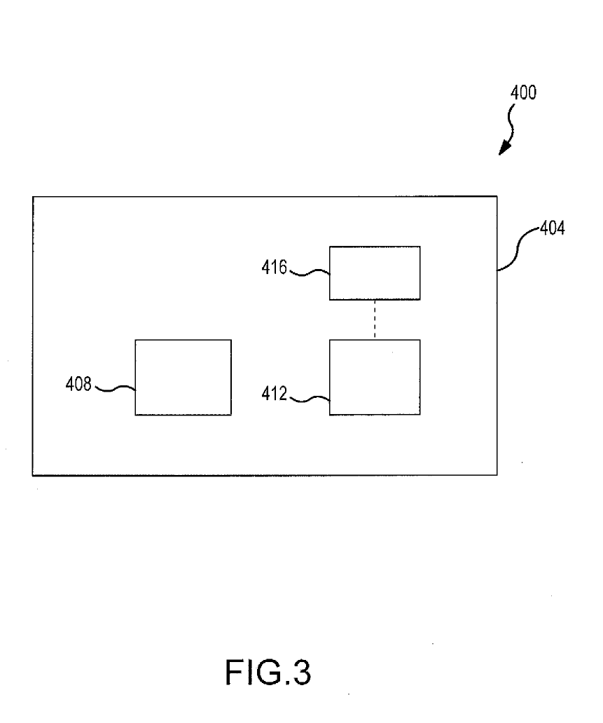

The embodiments of FIGS. 2D-2G are useful as part of a medical fluid delivery system in the environment of MRI equipment due to the absence of any ferrous components or incompatible magnetic fields in the embodiments. With reference to FIG. 3, an MRI suite 400 includes a room 404 shielded from electromagnetic interference in any appropriate manner (e.g., EMI shielded walls). One example of an MRI suite is disclosed in U.S. Pat. No. 7,512,434, and this patent is hereby incorporated by reference. The room 404 may include MRI imaging equipment 408 and an injection device 412 (e.g., power injector) that may use a piezoelectric-driven hydraulic syringe plunger drive assembly in accordance with any of the embodiments of FIGS. 2D-2G. The injection device 412 may be appropriately attached or mounted, for example, to a suspension arm 416, stand 48 (FIG. 2A), or other appropriate support device (not shown). It is contemplated that a portion or an entirety of one or more of the injection devices 412 of the embodiments described herein may be located within the electromagnetically shielded room 404 without adversely affecting the operation of the MRI imaging equipment 408 and without the MRI imaging equipment 408 adversely affecting the operation of the injection device 412.

While the components of some of the syringe plunger drive assemblies 56′, 132, 132′, 132″ of the various embodiments have been illustrated as being located within or entirely within the housings 192, 131, 131′, 131″, it is contemplated that one or more of the various components could be located outside of housings 192,131, 131′, 131″. For instance, the piezoelectric pump 178, 178′, 110 could be associated with or mounted within other components such as a pedestal, ceiling suspension arm, and the like. Similarly, it is contemplated that one or more of the components of the syringe plunger drive assemblies 56′, 132, 132′, 132″ of the various embodiments currently illustrated in the figures as being located outside of the housings 192, 131, 131′, 131″ could be located within the housings 192, 131, 131′, 131″. Moreover and although not shown, a sensor or multiple sensors may be associated with the syringe plunger drivers. Such sensors may be usable to determine the position of the ram coupler 76 (and therefore the position of an interconnected plunger 90a/b) and/or measure the linear distance traveled by the piston and piston rod. The sensor(s) may be in the form of a non-ferrous linear potentiometer; however, other appropriate types of non-ferrous sensors could also be incorporated into the syringe plunger drive assemblies.

Further, other arrangements of the piezoelectric pumps 110, 178, 178′ are contemplated. For instance, referring to the embodiment of FIG. 20, the piezoelectric pump 110 could include an interface that allows an operator and/or an interfacing controller (e.g., a controller of the powerhead 130) to input a desired output (e.g., desired flow rate and/or pressure, desired linear movement or stroke of the piston), and then a protocol or other device of the piezoelectric pump 110 would automatically determine the appropriate voltage and/or frequency to apply to the piezoelectric stack 112 to produce the desired output. In another embodiment, electrical leads could be attached directly to the external leads of the piezoelectric pump 110 to allow direct control of the piezoelectric stack 112 by an external controller (e.g., a controller of the powerhead 130). Further, while each of the above piezoelectric pumps 110, 178, 178′ may be a PHP 2 Piezo Hydraulic Pump developed by Kinetic Ceramics, Inc. of Hayward, Calif., it will be recognized that other types of piezoelectric pumps usable in an MRI suite could also be used with the syringe plunger drive assemblies 132, 132′, 132″ and 56′. It will also be appreciated that any of the pumps 110, 178, 178′, valves or interconnecting assemblies (not labeled) may be utilized with any of the embodiments described herein.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain best modes known of practicing the invention and to enable others skilled in the art to utilize the invention in such or other embodiments and with various modifications required by the particular application(s) or use(s) of the present invention. It is intended that the appended claims be construed to include alternative embodiments to the extent permitted by the prior art.

1. A medical fluid delivery system, comprising:
an injection device comprising a first syringe plunger driver and a piezoelectric pump, wherein said piezoelectric pump is operatively interconnected to said first syringe plunger driver for driving said first syringe plunger driver in at least a first direction; and
a syringe comprising a syringe barrel and a syringe plunger, wherein said first syringe plunger driver interacts with said syringe plunger to move said syringe plunger in at least said first direction within and relative to said syringe barrel.

2. The system of claim 1, wherein said first syringe plunger driver comprises a hydraulic cylinder.

3. The system of claim 2, wherein said hydraulic cylinder is single acting.

4. The system of claim 3, wherein said hydraulic cylinder comprises a piston, a piston rod and at least one biasing member, wherein said at least one biasing member is disposed to urge said piston and said piston rod in a second direction opposite said first direction.

5. The system of claim 4, wherein said piezoelectric pump comprises an inlet port and an outlet port and said hydraulic cylinder comprises a first port, and wherein hydraulic fluid from said outlet port enters said first port to move said piston and said piston rod in said first direction.

6. The system of claim 5, further comprising a valve movable between an at least partially open position and a closed position, wherein when said valve is in said at least partially open position, fluid is operable to flow out of said first port, and wherein when said valve is in said closed position, fluid is substantially not operable to flow out of said first port.

7. The system of claim 2, wherein said hydraulic cylinder is double acting.

8. The system of claim 7, wherein said piezoelectric pump comprises an inlet port and an outlet port and said hydraulic cylinder comprises a first port and a second port, said system further comprising valving operable to selectively fluidly interconnect:
said inlet port to said first port and said outlet port to said second port in a first configuration; and
said inlet port to said second port and said outlet port to said first port in a second configuration.

9. The system of claim 1, wherein said first syringe plunger driver comprises a hydraulic motor.

10. The system of claim 9, wherein said first syringe plunger driver comprises a ram movable on a drive screw, and wherein said hydraulic motor assists in rotating said drive screw to move said ram along said drive screw to move said syringe plunger.

11. The system of claim 10, further comprising a driving gear mounted to said hydraulic motor and a driven gear mounted on said drive screw, wherein rotation of said driving gear rotates said drive screw.

12. The system of claim 1, wherein said injection device comprises a housing, and wherein said piezoelectric pump and said first syringe plunger driver are located at least partially within said housing.

13. The system of claim 1, wherein said injection device is attached to one of a pedestal and a suspension arm.

14. The system of claim 1, further comprising a reservoir, wherein said first syringe plunger driver is selectively fluidly interconnectable to said reservoir and said piezoelectric pump is fluidly interconnected to said reservoir.

15. The system of claim 1, further comprising at least one piezoelectric valve.

16. The system of claim 1, wherein said piezoelectric pump comprises a pumping chamber and a piezoelectric stack, and wherein said piezoelectric stack is operable to expand to perform one of expelling hydraulic fluid from said pumping chamber and drawing hydraulic fluid into said pumping chamber, and to contract to perform the other of expelling hydraulic fluid from said pumping chamber and drawing hydraulic fluid into said pumping chamber.

17. The system of claim 16, wherein said piezoelectric pump is operable to drive said first syringe plunger driver in said first direction when said piezoelectric stack expands.

18. The system of claim 1, wherein said injection device further comprises:
a second syringe plunger driver, wherein said piezoelectric pump is operatively interconnected to said second syringe plunger driver for driving said second syringe plunger driver in at least a corresponding first direction; and
a second syringe comprising a second syringe barrel and a second syringe plunger, wherein said second syringe plunger driver interacts with said second syringe plunger to move said second syringe plunger in at least said corresponding first direction within and relative to said second syringe barrel.

19. The system of claim 18, further comprising a selector valve operable to fluidly interconnect said first syringe plunger driver to said piezoelectric pump in a first position of said selector valve and said second syringe plunger driver to said piezoelectric pump in a second position of said selector valve.

20. The system of claim 1, wherein said injection device further comprises:
a second syringe plunger driver, wherein said piezoelectric pump is operatively interconnected to said second syringe plunger driver for driving said second syringe plunger driver in at least a corresponding first direction; and
a second syringe comprising a second syringe barrel and second syringe plunger, wherein said second syringe plunger driver interacts with said second syringe plunger to move said second syringe plunger in at least said corresponding first direction within and relative to said second syringe barrel;
a selector valve operable to fluidly interconnect said first syringe plunger driver to said piezoelectric pump in a first position of said selector valve and said second syringe plunger driver to said piezoelectric pump in a second position of said selector valve, wherein said piezoelectric pump comprises an inlet port and an outlet port, and each of said first and second syringe plunger drivers comprises a first port and a second port; and
a directional valve movable between first and second positions; wherein said first position of said directional valve is operable to fluidly interconnect said inlet port to said first port of one of said first or second syringe plunger drivers and said outlet port to said second port of said one of said first or second syringe plunger drivers; and wherein said second position of said directional valve is operable to fluidly interconnect said inlet port to said second port of said one of said first or second syringe plunger drivers and said outlet port to said first port of said one of said first or second syringe plunger drivers.

21. A magnetic resonance imaging suite, comprising:
a room shielded from electromagnetic interference and comprising at least one magnetic resonance imaging device; and
the system of claim 1, wherein an entirety of said system is located within said room.
